(19) United States
(12) Patent Application Publication
(10) Pub. No.: US 2008/0175898 A1
(43) Pub. Date: Jul. 24, 2008

(54) ORAL DRUG CAPSULE COMPONENT INCORPORATING A COMMUNICATION DEVICE

(75) Inventors: Christopher Michael Jones, Midland, MI (US); Douglas Paul White, Lake Jackson, TX (US); Peter K. Mercure, Midland, MI (US)

Correspondence Address: BUTZEL LONG;IP DOCKETING DEPT, 350 SOUTH MAIN STREET, SUITE 300, ANN ARBOR, MI, 48104, US

(73) Assignee: Dow Global Technologies, Inc., Midland, MI (US)

(21) Appl. No.: 12/015,201

(22) Filed: Jan. 16, 2008

Related U.S. Application Data
(60) Provisional application No. 60/880,606, filed on Jan. 16, 2007.

(51) Int. Cl. A61K 9/52

(52) U.S. Cl. 424/452

(57) ABSTRACT

An improved upper capsule portion (62) of an oral drug delivery capsule (60) that includes an upper capsule portion (62) and a lower cup shaped capsule portion (64), the lower cup shaped capsule portion (64) containing a medical formulation (66), the lower capsule portion (64) being made of a material that disperses in gastrointestinal fluid, the lower capsule portion (64) having a mouth, the upper capsule portion (62) dimensioned to engage with the mouth of the lower capsule portion (64). The improvement is the positioning of a communication device, such as an RFID tag (90), on or integrally with the upper capsule portion (62) so that the communication device can communicate that the oral drug delivery capsule has been ingested. An alternate embodiment with an improved lower capsule portion is also disclosed.

### BACKGROUND OF THE INVENTION

The present invention relates to a method and system for monitoring compliance to an internal dosing regimen and the subsequent analysis of the data generated. More particularly, the present invention relates to the use of an ingested or inserted encapsulated device that delivers a signal to an external data collection device for observation and analysis when a switch sensitive to the ionically conductive environment of the gastrointestinal tract is triggered, thereby indicating that the dose form has been ingested, inserted or otherwise internalized. The data collected in the external data collection device may then be analyzed for management of patient therapy or for clinical study.

Non-compliance refers to the failure by the patient to take the prescribed dosage at the prescribed time for the prescribed period, resulting in patient under-medication or over-medication. Such non-compliance results in increased cost of medical care, higher complication rates, higher rates of drug-resistance by pathogens, and drug wastage. In a survey of 57 non-compliance studies, failure to comply with the drug regimen ranged from 15% to as high as 95% in all study populations, regardless of medications, patient population characteristics, the drug being delivered, or study methodology. (Greeberg, R. N.: Overview of Patient Compliance with Medication Dosing: A Literature Review, Clin. Therap., 6(5):592-599 [1984].) Reasons for the failure of patients to comply with drug regimens are plentiful and include forgetfulness (30%), other matters taking priority (16%), choosing not to take drug (11%), lack of information (9%) and “emotional factors” (7%). (Osterberg, L., and Blaschke, T.: Compliance to medication, N. Engl. J. Med. 353;5, 490 [2005].)

Compliance to the instructions given to patients during any clinical trial is usually less than 50% in relatively short-term and less than 40% in longer-term trials using traditional methods (e.g., paper diaries) for making entries to show compliance (Vrijens and Goetghebeur, Statist. Med. 23, 531-544, 2004). A clinical trial on chronic pain patients reported only an 11% compliance with as high as 80% fake entries when paper diaries secretly instrumented to track diary usage were given to patients (Stone et al., Control Clin. Trials. 24, 182-199, 2003) wherein on 32% of study days the paper diary was not opened, yet the compliance entries for those days exceeded 90%. A high incidence of intentional dumping of medications prior to the clinic visit by removing all or most of the medication at one time also occurs in clinical studies (Coutts et al, Arch. Dis. Child. 67, 332-333, 1992; Rand et al, Am. Rev. Respir. Dis. 146, 1559-1564, 1992; Rudd et al, Clin. Pharmacol. Therap. 46, 169-176, 1989; Simmons et al, Chest. 118, 290-295, 2000). Thus, deception among noncompliant patients occurs frequently in clinical trials, and is not often revealed by the traditional monitoring methods. The result is generation of data difficult to interpret and, worse, useless to reliably predict the effectiveness of clinical trials. Better monitoring of the time of actual drug intake will help alleviate many of these issues. For example, blood levels of a drug can be corrected for the time of actual drug intake for better pharmacokinetic/pharmacodynamic interpretations than relying on the time when patient(s) was instructed to take the medication. However, most of the present tracking devices that are utilized in clinical trials only track the initiation of the process of drug intake, i.e., by tracking the time the drug containers are opened or activated. In order to more accurately monitor the compliance of a clinical trial, a more sophisticated method of monitoring the drug intake is needed.

In the therapeutic setting, accurately measuring and analyzing compliance has a number of important benefits such as enabling the care-giver to warn a patient about the potential for developing a drug resistant infection related to poor compliance to the regimen and enabling the identification of a side effect of a drug related to overdosing. In the clinical drug research stage, accurately measuring and analyzing compliance can lead to a broad range of benefits, including improved statistical reliability of a clinical study, earlier completion of clinical studies, possible identification of side effects, and a determination of the effects of non-compliance as a function of the degree of non-compliance.

Confirmation of drug compliance by way of direct observation by trained persons is effective but impractical in most settings. Confirmation of drug compliance by blood or urine analysis is also not practical beyond the hospital setting.

There have been technical efforts made to overcome the impracticality of direct observation and specimen analysis. These technical efforts have been singularly directed to monitoring dosing compliance. Trans-dermal detection devices attached to the skin of a patient have been developed which detect ingested drug components through the skin. Such devices can transmit a signal to a remote receiver at an external site such as a healthcare facility as disclosed in, for example, U.S. Pat. No. 6,663,846 and U.S. Published Patent Application No. 2005/0031536. Electronic sensor systems have also been developed which detect ingested drug components in the breath of a patient, such as set forth in U.S. Published Patent Application No. 2004/0081587. Radio Frequency Identification (“RFID”) tags have been incorporated into pills with each tag capable of identifying the type of medication, its dosage, and its lot number by way of a unique code emitted by the tag when interrogated by a corresponding radio frequency reader, as set forth in U.S. Pat. No. 6,366,206. The RFID of the '206 patent can incorporate a biosensor that switches state, for example, by detecting ionic conductivity, in the gastrointestinal tract detects moisture or change in pH to determine whether the pill has dissolved and exposed the RFID tag to the environment of the gastrointestinal system.

Statistical models for drug compliance have also been developed. For example, Gerard et al. in Statistics in Medicine (17, 2313-2333 [1998]) describe a Markov mixed effect model for drug compliance data. Vrijens et al., in Statistics in Medicine (23, 531-544 [2004]), describe a data treatment model for reduced bias and improved precision in pharmacokinetic pharmacodynamic population studies. In European Patent Application No. 0526166 a patient compliance monitoring method using a radio transmitter attached to a medicine container to detect medicine consumption is disclosed. A patient compliance monitoring method based on patient entry of data related to medicine consumption is disclosed in U.S Published Patent Application No. 2002/0143577.

A bar code-based drug dispensing system and database are disclosed in U.S Published Patent Application No. 200310055531. In U.S Published Patent Application No. 2003/0110060, a patient compliance monitoring method that includes interaction with the patient is disclosed. A patient compliance monitoring system which provides the patient with a portable medication dispenser which alerts the patient to take a dose of medication and then gathers compliance data relating to the taking of the medication is set forth in U.S Published Patent Application No. 2004/0133305.

A patient compliance monitoring method employing a pharmacokinetic model to determine if the prescribed dosing regimen should be adjusted is provided in U.S Published Patent Application No. 2004/01193446. The use of a patient compliance monitoring method for use in clinical trials is disclosed in U.S Published Patent Application No. 2004/0243620. A system and method for tracking drug containers is disclosed in U.S Published Patent Application No. 2004/0008123. Finally, a patient compliance monitoring method employing a capsule or pill containing an RFID tag which is responsive to ingestion by a patient is disclosed in U.S Published Patent Application No. 2005/0131281.

Each of the above-described patents and publications provides a contribution to the state of the art with respect to monitoring compliance to a dosing regimen. However, as in so many areas of art, there is room for improvement in the monitoring of an internal dosing regimen.

### SUMMARY OF THE INVENTION

The instant invention is an improved means of incorporating an RFID tag or other communication device, with a drug delivery capsule. More specifically, the instant invention is an improved upper capsule portion of an oral drug delivery capsule comprised of the upper capsule portion and a lower cup shaped capsule portion, the lower cup shaped capsule portion for containing a medical formulation, the lower capsule portion being made of a material that disperses in gastrointestinal fluid, the lower capsule portion having a mouth, the upper capsule portion dimensioned to engage with the mouth of the lower capsule portion, wherein the improvement comprises: a communication device positioned on or integrally with the upper capsule portion so that the communication device can communicate that the oral drug delivery capsule has been ingested.

In a related embodiment, the instant invention is an improved lower capsule portion of an oral drug delivery capsule comprised of the upper capsule portion and a lower cup shaped capsule portion, the lower cup shaped capsule portion for containing a medical formulation, the lower capsule portion being made of a material that disperses in gastrointestinal fluid, the lower capsule portion having a mouth, the upper capsule portion dimensioned to engage with the mouth of the lower capsule portion, wherein the improvement comprises: a communication device positioned on or integrally with the lower capsule portion so that the communication device communicates that the oral drug delivery capsule has been ingested.

### BRIEF DESCRIPTION OF THE DRAWINGS

For a more complete understanding of this invention, reference should now be made to the embodiments illustrated in greater detail in the accompanying drawings and described below by way of examples of the invention wherein:

FIG. 1 is an enlarged view, part in cross-section and part in full, of a tamper proof oral drug delivery capsule having an upper capsule portion fitted in the mouth of the lower capsule portion, the upper capsule portion containing an active RFID tag;

FIG. 2 is an enlarged view, part in cross-section, part broken away and part in full, of an oral drug delivery capsule having an upper capsule portion fitted over the mouth of the lower capsule portion with a passive RFID tag system wrapped on and adhered to the lower capsule portion;

FIG. 3 is an enlarged view, part in cross-section and part in full, of an oral drug delivery capsule having an upper capsule portion fitted in the mouth of the lower capsule portion, the upper capsule portion containing a magnet;

FIG. 4 is an enlarged view, part in cross-section and part in full, of a tamper proof oral drug delivery capsule having an upper capsule portion fitted in the mouth of the lower capsule portion, the upper capsule portion containing an infra-red emitting diode;

FIG. 5 is an enlarged view, part in cross-section and part in full, of an oral drug delivery capsule having an upper capsule portion fitted over the mouth of the lower capsule portion, the upper capsule portion containing a radio frequency transmitter system;

FIG. 6 is an enlarged view, part in cross-section and part in full, of an oral drug delivery tablet having adhered thereto an RFID tag system;

FIG. 7 is an enlarged view, part in cross-section and part in full, of a tamper proof oral drug delivery capsule having an upper capsule portion fitted in the mouth of the lower capsule portion, the upper capsule portion containing a fluorescent agent; and

FIG. 8 is an enlarged view, part in cross-section and part in full, of an oral drug delivery capsule having an upper capsule portion fitted inside the mouth of the lower capsule portion, the upper capsule portion containing an ultrasonic transducer.

### DETAILED DESCRIPTION

In the following figures, the same reference numerals will be used to refer to the same components. In the following description, various operating parameters and components are described for one constructed embodiment. These specific parameters and components are included as examples and are not meant to be limiting.

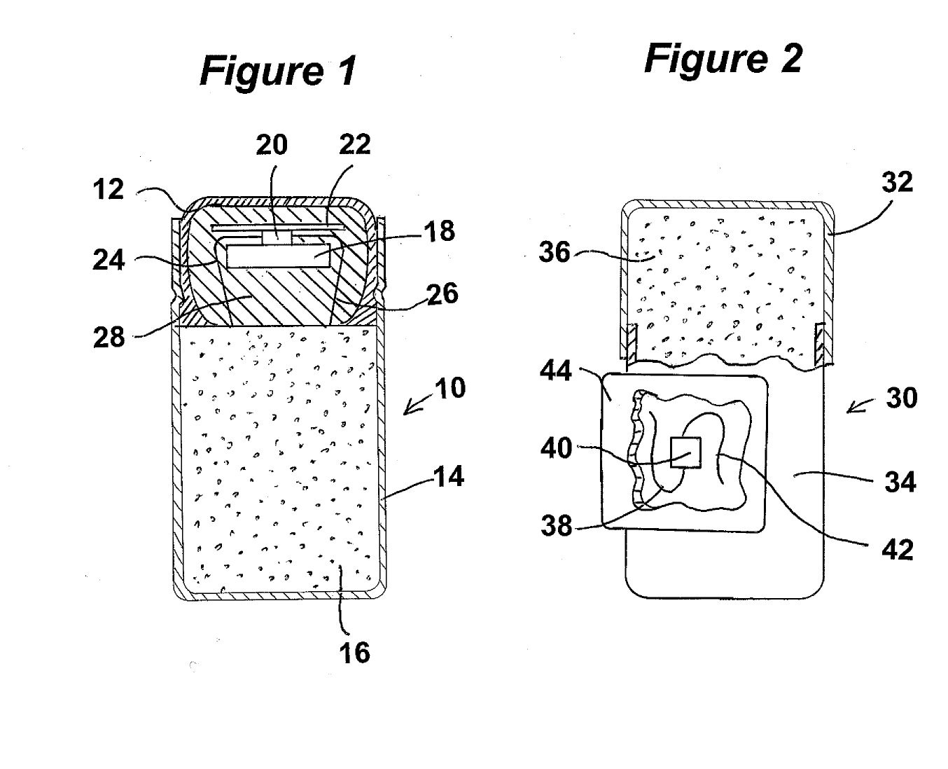

Referring now to FIG. 1, therein is shown a tamper proof oral drug delivery capsule 10 comprising an upper capsule portion made of a molded thermoset plastic core 28 overmolded with gelatin 12 and a lower capsule portion 14 made of gelatin. A drug formulation 16 is positioned in the lower capsule portion 14. The capsule 10 as illustrated is a capsule, but it is to be understood that other forms of dosing such as tablets and pills may be used as well. The dose form as used herein refers to a dose that includes an active drug ingredient or a may be a placebo.

An RFID chip 20 is positioned in the core 28. By way of non-limiting example, the RFID chip 20 may be coded to indicate, among other things, the type of medication, the dose of the medication and the lot and serial numbers of the medication. As set forth below, the capsule 10 emits a signal to indicate that the dose form 10 has, in fact, been ingested, based upon its having a switch activated by exposure to the gastrointestinal tract. The signal may be emitted in a variety of ways, including, as examples, electromagnetic (e.g., visible light, ultraviolet and infrared radiation, or an RFID signal), magnetic, radioactive, chemical (e.g., a tracer detectable on the breath), fluorescent, acoustic (e.g., ultrasonic or gasified candy-type technology), and biological (e.g., using biomarkers, as from the evolving area of tetramer technology).

The RFID chip 20 may be of any one of several designs and configurations. Accordingly, the RFID chip 20 as shown is for illustrative purposes only and is not intended as being limiting. The signal from the RFID chip 20 can be amplified by a signal amplifier positioned between the RFID chip 20 and a signal-receiving and reading device (neither shown).

The RFID chip 20 is attached to an antenna 22 and a battery 18. When the capsule 10 is ingested, the lower capsule portion 14 disperses in gastric fluid and electrodes 24 and 26 are exposed to the gastric fluid. Electrodes 24 and 26 are attached at one end thereof to the RFID chip 20 and comprise a conductivity switch incorporated in RFID chip 20 to turn on the RFID chip 20 when the capsule 10 is ingested thereby exposing the electrodes 24 and 26 to electrically conducting gastric fluid.

Referring now to FIG. 2, therein is shown an oral drug delivery capsule 30 comprising an upper capsule portion 32 made of gelatin and a lower capsule portion 34 made of gelatin. A drug formulation 36 is positioned in the capsule portions 32 and 34. A passive RFID chip 40 is positioned in a patch 44 wrapped on and adhered to the lower capsule portion 34. The RFID chip 40 is encoded to identify a drug type, dose, lot number etc. The RFID chip 40 is attached to dipole antennae 38 and 42. When the capsule 30 is ingested, the capsule portions 32 and 34 disperse in gastric fluid and RFID chip 40 is warmed to body temperature. RFID chip 40 contains a thermal switch to turn on the RFID chip 40 when the capsule 30 is ingested and the RFID chip 40 is warmed to body temperature.

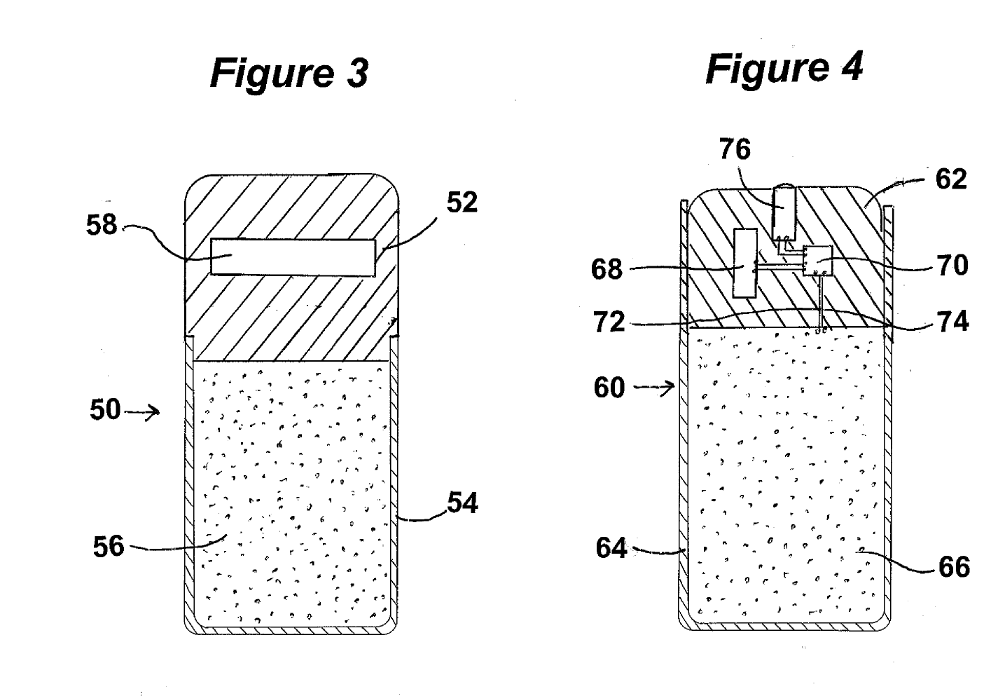

Referring now to FIG. 3, therein is shown an oral drug delivery capsule 50 comprising an upper capsule portion 52 made of a molded thermoplastic and a lower capsule portion 54 made of gelatin. A drug formulation 56 is positioned in the lower capsule portion 54. A magnet 58 is positioned in the upper capsule portion 52. When the capsule 50 is ingested, the presence of the magnet 58 is detected by a magnetometer contained in an article that can be placed on or worn by the user, such as a necklace.

Referring now to FIG. 4, therein is shown a tamper proof oral drug delivery capsule 60 comprising an upper capsule portion 62 made of a molded thermoset plastic and a lower capsule portion 64 made of gelatin. A drug formulation 66 is positioned in the lower capsule portion 64. A microprocessor 70 is positioned in the upper capsule portion 62. The microprocessor 70 is encoded to identify a drug type, dose, lot number etc. The microprocessor 70 is attached to an infrared diode 76 and a battery 68. When the capsule 60 is ingested, the lower capsule portion 64 disperses in gastric fluid and electrodes 72 and 74 are exposed to the gastric fluid. Electrodes 72 and 74 are attached at one end thereof to the microprocessor 70 and comprise a conductivity switch incorporated in microprocessor 70 to energize the infrared diode 76 in a modulated encoded manner when the capsule 40 is ingested thereby exposing the electrodes 72 and 74 to electrically conducting gastric fluid. The emitted infrared radiation from the diode 76 is detected by an infrared detector contained in a pouch worn around the abdomen.

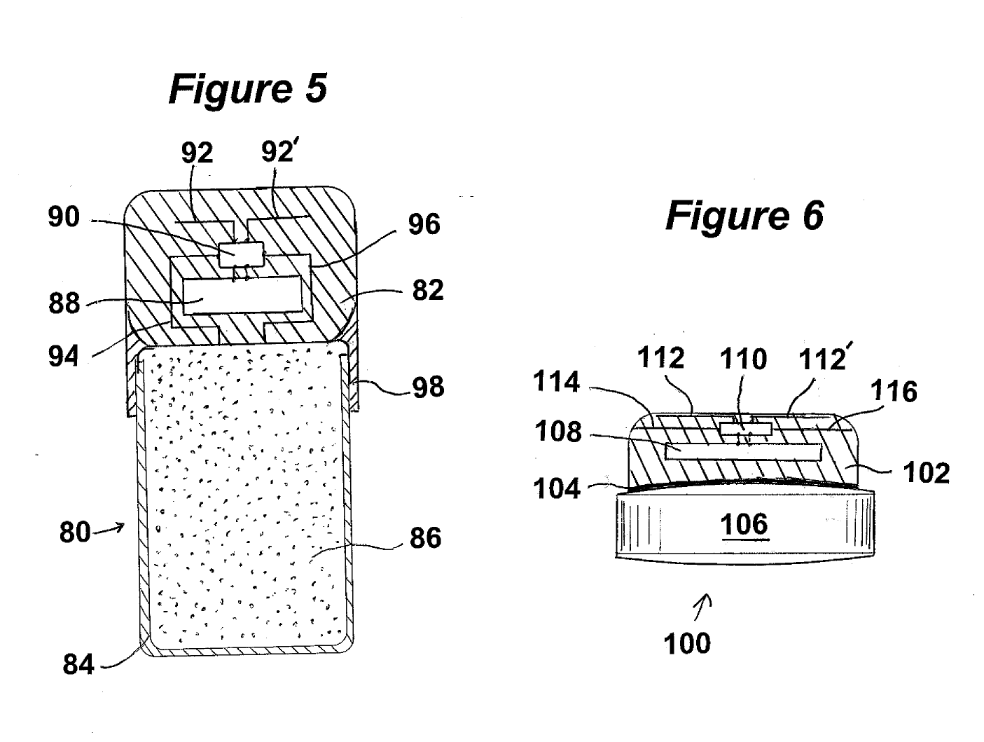

Referring now to FIG. 5, therein is shown an oral drug delivery capsule 80 comprising an upper capsule portion made of a molded thermoset plastic core 82 attached to a gelatin skirt 98 and a lower capsule portion 84 made of gelatin. A drug formulation 86 is positioned in the lower capsule portion 84. A radio frequency generator 90 is positioned in the core 82. The specific frequency of the radio frequency generator 90 identifies a drug type, dose, lot number etc. The radio frequency generator 90 is attached to an antenna 92 and a battery 88. When the capsule 80 is ingested, the lower capsule portion 84 disperses in gastric fluid and electrodes 94 and 96 are exposed to the gastric fluid. Electrodes 94 and 96 are attached at one end thereof to radio frequency generator 90 and comprise a conductivity switch incorporated in radio frequency generator 90 to turn on the radio frequency generator 90 when the capsule 80 is ingested thereby exposing the electrodes 94 and 96 to electrically conducting gastric fluid.

Referring now to FIG. 6, therein is shown an oral drug delivery tablet system 100. An active RFID chip 110 is positioned in a molded thermoplastic body 102 bonded to a drug delivery tablet 106 by a layer of adhesive 104. The RFID chip 110 is encoded to identify a drug type, dose, lot number etc. The RFID chip 110 is attached to antennae 112, 112′ and a battery 108. When the tablet 100 is ingested electrodes 114 and 116 are exposed to the gastric fluid. Electrodes 114 and 116 are attached at one end thereof to the RFID chip 110 and comprise a conductivity switch incorporated in RFID chip 110 to turn on the RFID chip 110 when the tablet 100 is ingested thereby exposing the electrodes 114 and 116 to electrically conducting gastric fluid.

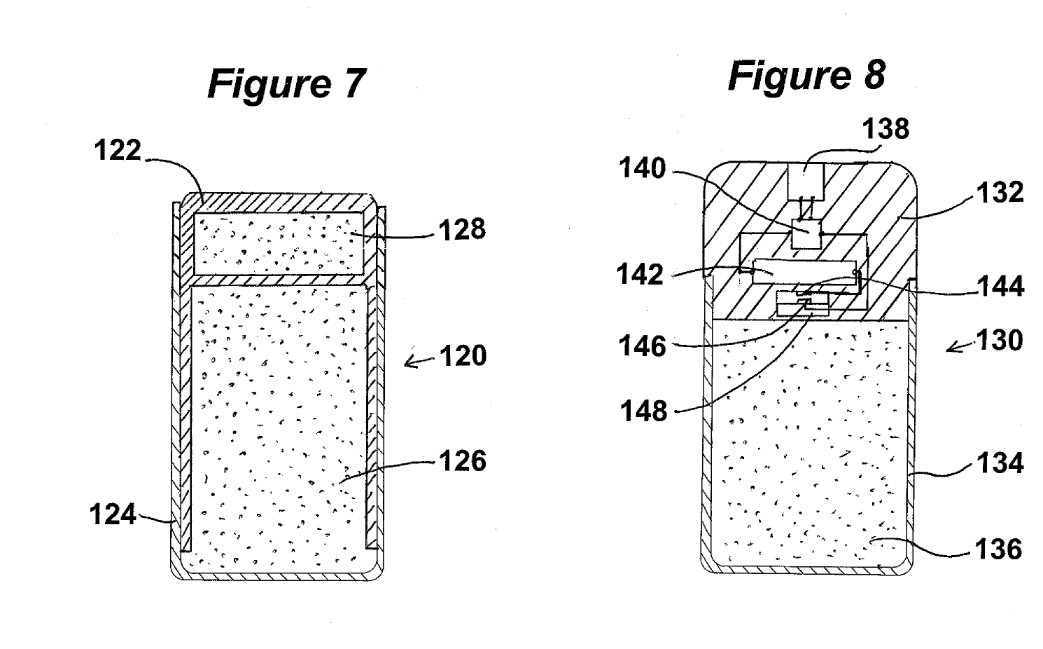

Referring now to FIG. 7, therein is shown a tamper proof oral drug delivery capsule 120 comprising a lower capsule portion 124 made of gelatin and an upper capsule portion 122 also made of gelatin. A drug formulation 126 is positioned in the lower capsule portion 124. A fluorescing reagent 128 is positioned in the upper capsule portion 122. When the tamper proof oral drug delivery capsule 120 is ingested, the upper and lower capsule portions disperse in the gastrointestinal system thereby allowing the fluorescing reagent 128 to enter the blood stream to be detected by a fluorescence detector positioned on the skin.

Referring now to FIG. 8, therein is shown an oral drug delivery capsule 130 comprising an upper capsule portion 132 made of a molded thermoset plastic and a lower capsule portion 134 made of gelatin. A drug formulation 136 is positioned in the lower capsule portion 134. A microprocessor 140 is positioned in the upper capsule portion 132. The microprocessor 140 is encoded to identify a drug type, dose, lot number etc. The microprocessor 140 is attached to an ultrasonic transducer 138 and one pole of battery 142. The other pole of battery 142 is connected to first electrical contact 144. Second electrical contact 146 is connected to microprocessor 140. Second electrical contact 146 is positioned on pad 148 made of a material that swells upon exposure to gastric fluid. When the capsule 130 is ingested, pad 148 swells upon exposure to gastric fluid and causes second electrical contact 146 to contact first electrical contact 144 thereby turning on ultrasonic transducer 138 in a modulated encoded manner. The emitted ultrasonic radiation from the transducer 138 is detected by an ultrasonic detector contained in a pouch worn around the abdomen.

The lower capsule portion of the instant invention can be made of any material that disperses in gastrointestinal fluid, such as gelatin, hydroxypropylmethylcellulose and poly-N,N-9-diethylaminoethyl methacrylate. The upper capsule portion can be made of any suitable material, such as molded thermoplastic polymer such as polyethylene, polypropylene, polystyrene and polycarbonate or molded thermoset polymer such as an epoxy resin or a urethane polymer.

The specific means of detecting the communication device is not critical in the instant invention. The detection system (such as an RFID reader when the communication device is an RFID tag) in communication with the communication device is preferably battery powered and positioned on or near the person, preferably in a watch-like device worn on the wrist, in a necklace-like device worn around the neck, in a device worn on or near the abdomen or in a patch worn on the skin. The detection system is preferably programmed to sense and record the type of drug(s) and times of administration thereof for later downloading or preferably for wireless downloading to, for example, healthcare professionals who could even send a reminder signal to the system to remind the patient of his/her noncompliance.

When the communication device used in the instant invention is an RFID tag, then it should be understood that any type of RFID tag can be used, including active and passive RFID tags (passive RFID tags are preferred). Although several specific and preferred means of sensing ingestion are described above, it should be understood that any means can be used to sense ingestion including all of the means disclosed in U.S. Ser. No. 11/436,917 filed May 18, 2006, herein fully incorporated by reference.

Although FIGS. 1, 4 and 7 refer to specific tamper-proof capsule embodiments, it should be understood that any tamper-proof capsule design can be used in the instant invention, including the designs of U.S. Pat. No. 4,893,721, herein fully incorporated by reference. In addition, the oral drug capsule of the present invention can be used with a variety of systems, such as that disclosed in U.S. Ser. No. 11/693,404, filed Mar. 29, 2007, herein fully incorporated by reference.

### EXAMPLE

An oral drug delivery capsule like the capsule 10 of FIG. 1 is assembled. A 433 MHz active RFID tag having a conductivity switch is placed in the upper capsule portion while a simulated drug formulation consisting of food grade lactose is placed in the lower capsule portion. The capsule is placed in a plastic wire screen basket placed in the center of a 50 liter polyethylene tank containing 40 liters of USP Simulated Gastric Fluid at 37 degrees Celsius with agitation. A receiving dipole antenna is positioned at the bottom of the tank. Another receiving dipole antenna is positioned outside the tank. The gelatin capsule disperses in the simulated gastric fluid and the conductivity switch turns on the RFID tag which then transmits its 433 MHz signal. The signal strength received by the antenna in the tank is about 5 nanowatt. The signal strength received by the antenna outside the tank held against the tank is about 0.1 nanowatt. The signal strength received by the antenna outside the tank held 70 centimeters away from the tank is about 0.01 nanowatt. An arm held between the tank and the antenna slightly (2-3 dB) reduces the signal strength received by the antenna.

The minimum detectable signal strength received by the antenna outside the tank held even further from the tank is estimated to be about 0.0001 nanowatt. The signal strength received by the antenna outside the tank is only slightly dependent (a variation of about 1-5 dB) on the position of the antenna of the RFID tag.

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

What is claimed is:

1. An improved upper capsule portion of an oral drug delivery capsule comprising;
an upper capsule portion and a lower cup shaped capsule portion for containing a medical formulation, said lower capsule portion being made of a material that disperses in gastrointestinal fluid, said lower capsule portion having a mouth, said upper capsule portion being dimensioned to engage with said mouth of said lower capsule portion; and
a communication device positioned on or integrally with said upper capsule portion whereby said communication device can communicate that the oral drug delivery capsule has been ingested.

2. The improved upper capsule portion of claim 1, wherein said communication device is selected from the group consisting of an RFID tag, an electromagnetic signaling device, a magnetic device, an infrared emitting device and an ultrasonic device.

3. The improved upper capsule portion of claim 1, wherein the upper capsule portion is shaped so that the oral drug delivery capsule is tamper-proof.

4. The improved upper capsule portion of claim 2, wherein the upper capsule portion is shaped so that the oral drug delivery capsule is tamper-proof.

5. The improved upper capsule portion of claim 1 further comprising a receiver located on or near a person for communication with the communication device of the upper capsule portion.

6. The improved upper capsule portion of claim 2, further comprising a receiver located on or near a person for communication with the communication device of the upper capsule portion.

7. The improved upper capsule portion of claim 3, further comprising a receiver located on or near a person for communication with the communication device of the upper capsule portion.

8. An improved lower capsule portion of an oral drug delivery capsule comprising:
an upper capsule portion and a lower capsule portion, said lower capsule portion for containing a medical formulation, said upper capsule portion being made of a material that disperses in gastrointestinal fluid, said lower capsule portion having a mouth, said upper capsule portion being dimensioned to engage with said mouth of said lower capsule portion; and
a communication device positioned on or integrally with said lower capsule portion so that said communication device can communicate that the oral drug delivery capsule has been ingested.

9. The improved lower capsule portion of claim 5, wherein said communication device is selected from the group consisting of an RFID tag, an electromagnetic signaling device, a magnetic device, an infrared emitting device and an ultrasonic device.

10. The improved lower capsule portion of claim 8, wherein said upper capsule portion is shaped so that the oral drug delivery capsule is tamper-proof.

11. The improved lower capsule portion of claim 9, wherein said upper capsule portion is shaped so that the oral drug delivery capsule is tamper-proof.

12. The improved lower capsule portion of claim 8, further comprising a receiver located on or near a person for communication with said communication device of said lower capsule portion.

13. The improved lower capsule portion of claim 9, further comprising a receiver located on or near a person for communication with said communication device of said lower capsule portion.

14. The improved lower capsule portion of claim 10, further comprising a receiver located on or near a person for communication with said communication device of said lower capsule portion.

15. An improved oral drug delivery system, the oral drug delivery system selected from the group consisting of a capsule and a tablet, wherein the improvement comprises a communication device attached to the capsule or tablet so that the communication device communicates that the capsule or tablet has been ingested.

16. The improved oral delivery system of claim 15, wherein said communication device is selected from the group consisting of an RFID tag, an electromagnetic signaling device, a magnetic device, an infrared emitting device and an ultrasonic device.

17. The improved oral delivery system of claim 15, further including an upper capsule portion, said upper capsule portion being shaped so that the oral drug delivery capsule is tamper-proof.

18. The improved oral delivery system of claim 15, further including a lower capsule portion, said lower capsule portion being matable with said upper capsule portion.

19. The improved oral delivery system of claim 18, further comprising a receiver located on or near a person for communication with said communication device of said lower capsule portion.

20. The improved oral delivery system of claim 15 wherein said communication device further includes an antenna.
